(19) United States
(12) Patent Application Publication
(10) Pub. No.: US 2011/0166611 A1
(43) Pub. Date: Jul. 7, 2011

(54) COMBINATION DISTRACTER AND INSERTER INSTRUMENT

(75) Inventor: David T. Stinson, Woodinvile, WA (US)

(73) Assignee: CENTINEL SPINE, INC., NEW YORK, NY (US)

(21) Appl. No.: 12/941,147

(22) Filed: Nov. 8, 2010

Related U.S. Application Data
(60) Provisional application No. 61/259,600, filed on Nov. 9, 2009.

(51) Int. Cl. A61B 17/56

(52) U.S. Cl. 606/86 A; 606/90

(57) ABSTRACT

A combination distracter and inserter instrument is provided. The instrument may comprise a pair of paddles configured to be inserted in between vertebral bodies. Each paddle may be attached at one end to a control handle and terminate at an opposite end in a tapered tip. The instrument may include a movable arm positioned in between the elongate paddles. The movable arm is attached to the control handle and configured at an opposite end to hold an implant for insertion into a disc space between vertebral bodies. In use, the instrument distracts the vertebral bodies with the elongate paddles as the spinal fusion implant is being delivered to the disc space with the movable arm. The instrument may also include a feature that prevents overinsertion of the paddles into the patient's spine, and a feature that prevents overinsertion of the implant into the disc space.

### CROSS-REFERENCE TO RELATED APPLICATIONS

This application claims priority to U.S. Provisional No. 61/259,600, filed Nov. 9, 2009, and entitled “COMBINATION DISTRACTER AND INSERTER INSTRUMENT,” which is herein incorporated by reference in its entirety.

### FIELD

The present invention relates to orthopedic implants, and more particularly, to tools for implanting spinal implants that facilitate fusion of bone segments and associated methods. Even more particularly, the invention relates to a combination distracter and inserter instrument for a spinal fusion device.

### BACKGROUND

The integrity of the spine, including its subcomponents like the vertebral bodies and intervertebral discs that are well known structural body parts forming the spine, are key to a patient's health. These parts may become crushed or damaged as a result of trauma or injury, or damaged by disease (e.g., by tumor, autoimmune disease) or as a result of wear over time or degeneration caused by the normal aging process.

In many instances, one or more structural body parts can be repaired or replaced with a prosthesis or implant. For example, specific to the spine, one method of repair is to remove the vertebra (in whole or in part) and/or the disc (in whole or in part) and replace it with an implant or prosthesis. In some cases, it is necessary to stabilize a weakened or damaged spinal region by reducing or inhibiting mobility in the area to avoid further progression of the damage and/or to reduce or alleviate pain caused by the damage or injury. In other cases, it is desirable to join together the damaged vertebrae and/or induce healing of the vertebrae. Accordingly, an implant or prosthesis may be configured to facilitate fusion between two adjacent vertebrae. The implant or prosthesis may be placed without attachment means or fastened in position between adjacent structural body parts (e.g., adjacent vertebral bodies.)

Typically, an implant or prosthesis is secured directly to a bone structure by mechanical or biological means. One manner of spine repair involves attaching a fusion implant or prosthesis to adjacent vertebral bodies using a fixation element, such as a screw. Most implants and their attachment means are configured to provide an immediate, rigid fixation of the implant to the implantation site. In certain situations, the implantation process for the fusion implant can become cumbersome since multiple tools are required. It is therefore desirable to provide a delivery tool that facilitates ease of insertion of these types of spinal fusion implants, and consequently reduces the overall time in the operating room for the patient.

Although the following discussion focuses on spinal implants or prostheses, it will be appreciated that many of the principles may equally be applied to other structural body parts within a human or animal body.

### SUMMARY

The present disclosure provides a combination distracter and inserter instrument for a spinal fusion implant that allows a surgeon to distract the vertebral bodies between which the implant is to be placed, and also insert the implant into that site.

In accordance with one exemplary embodiment, a combination distracter and inserter instrument is provided. The instrument may comprise a pair of elongate paddles configured to be inserted in between a pair of adjacent vertebral bodies. Each paddle may be attached at one end to a control handle and terminate at an opposite end in a tapered tip. The instrument may also include a movable arm positioned in between the elongate paddles. The movable arm may be attached at one end to the control handle and configured at an opposite end to hold a spinal fusion implant for insertion into a disc space between the adjacent vertebral bodies. The movable arm may reside between the elongate paddles. In use, the instrument distracts the vertebral bodies with the elongate paddles as the spinal fusion implant is being delivered to the disc space with the movable arm. The instrument may also include a feature that prevents overinsertion of the paddles into the patient's spine, and a feature that prevents overinsertion of the implant into the disc space.

In accordance with another exemplary embodiment, a method for inserting a spinal fusion implant is provided. The method includes the steps of providing a spinal fusion implant, providing an insertion instrument for delivering the spinal fusion implant, and inserting the spinal fusion implant with the insertion instrument. The instrument may comprise a pair of elongate paddles configured to be inserted in between a pair of adjacent vertebral bodies. Each paddle may be attached at one end to a control handle and terminate at an opposite end in a tapered tip. The instrument may also include a movable arm positioned in between the elongate paddles. The movable arm may be attached at one end to the control handle and configured at an opposite end to hold a spinal fusion implant for insertion into a disc space between the adjacent vertebral bodies. The movable arm may reside between the elongate paddles.

In one step the control handle can be actuated to effect translational movement of the movable arm, and subsequent opening or closing of the elongate paddles. When the movable arm is translated towards the tapered tips of the elongate paddles, the elongate paddles open and distract the vertebral bodies. When the movable arm is translated away from the tapered tips of the elongate paddles, the elongate paddles close.

It is to be understood that both the foregoing general description and the following detailed description are exemplary and explanatory only and are not restrictive of the disclosure. Additional features of the disclosure will be set forth in part in the description which follows or may be learned by practice of the disclosure.

### BRIEF DESCRIPTION OF THE DRAWINGS

The accompanying drawings, which are incorporated in and constitute a part of this specification, illustrate several embodiments of the disclosure and together with the description, serve to explain the principles of the disclosure.

FIG. 1A illustrates a perspective view of a combination distracter and inserter instrument of the present invention.

FIG. 1B illustrates a side view of the instrument of FIG. 1A.

FIG. 1C illustrates a front perspective view of the instrument of FIG. 1A.

FIG. 2A illustrates a side view of the instrument of FIG. 1A in a closed position.

FIG. 2B illustrates a side view of the instrument of FIG. 1A moving into an open position.

FIG. 2C illustrates a side view of the instrument of FIG. 1A with a spinal fusion implant inserted into the intervertebral disc space.

FIG. 3 illustrates another perspective view of the instrument and spinal fusion implant of FIG. 2C.

### DESCRIPTION OF THE EMBODIMENTS

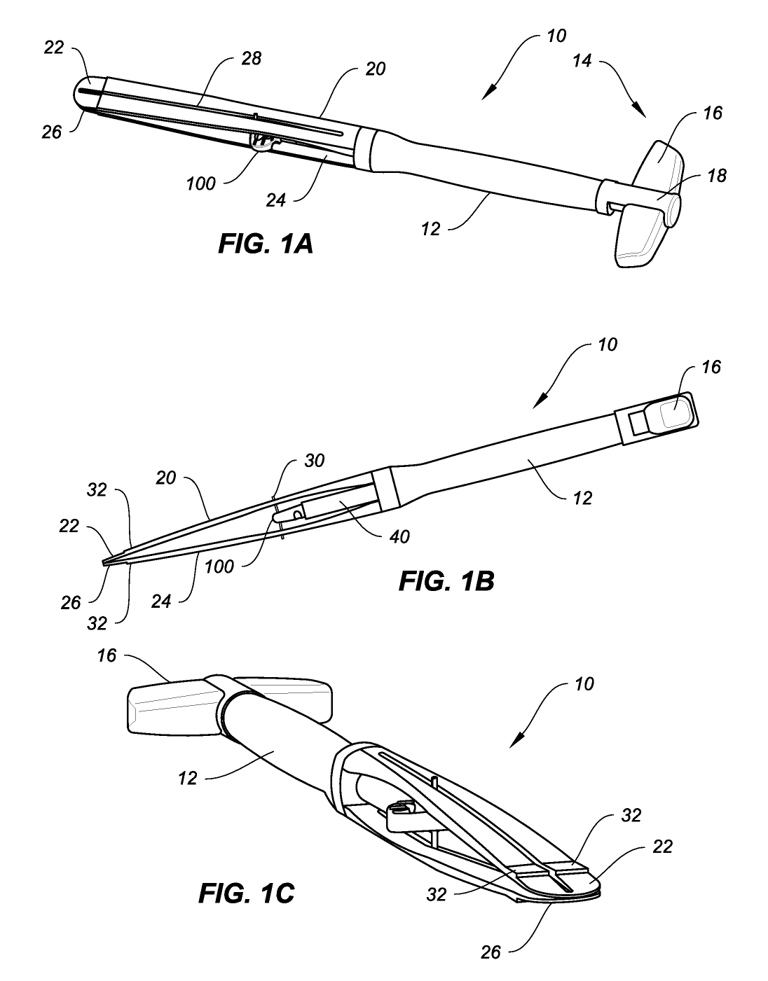

The present disclosure provides a combination distracter and inserter instrument for a spinal fusion implant that allows a surgeon to distract the vertebral bodies between which the implant is to be placed, and also insert the implant into that site. As shown in FIG. 1A, the instrument 10 comprises a shaft portion 12 connected to a pair of elongate paddles 20, 24. The elongate paddles 20, 24 extend into a tapered tip region 22, 26, respectively. The shaft portion 12 connects at an opposite end to a handle portion 14 that comprises a T-shaped grip 16 having a rotatable barrel 18. The paddles 20, 24 may have guide slots 28 that guide the movement of the implant 100 in/out of a desired position. The paddles 20, 24 may further include depth gauges or notches 32 comprising a surface feature such as a raised portion that serves as an insertion stop when the instrument 10 is in the open position, and to enhance the gripping surface of the paddles 20, 24. (See also FIG. 1C).

In the embodiments shown, the instrument 10 is shown as a substantially linear device. However, in other embodiments, the instrument 10 may be curved or angled (not shown) if desired by the user or surgeon to facilitate access or visibility to the vertebral bodies. For example, in other embodiments, the instrument 10 may comprise a handle 12 having a gearing arrangement that permits handle 12 to extend to angled positions, such as about 30, 45, or 90 degrees, etc., while still translating rotation of T-shaped grip 16 into linear motion of the implant 100. The handle 12 may be provided with a preset angle or an adjustable angle. Such gearing arrangements are known to those skilled in the art.

FIG. 1B shows a side view of the instrument 10. In this view, a movable arm, or motion arm 40 can be seen attached to the implant 100. Additionally, guide posts 30 may be provided on the motion arm 40. The guide posts 30 are configured to extend out through the guide slots 28 of the elongate paddles 20, 24 and may help maintain alignment of the motion arm 40 through its range of motion. FIG. 1C shows the instrument 10 in a closed position, where the tip region 22, 26 of the paddles 20, 24 touch one another.

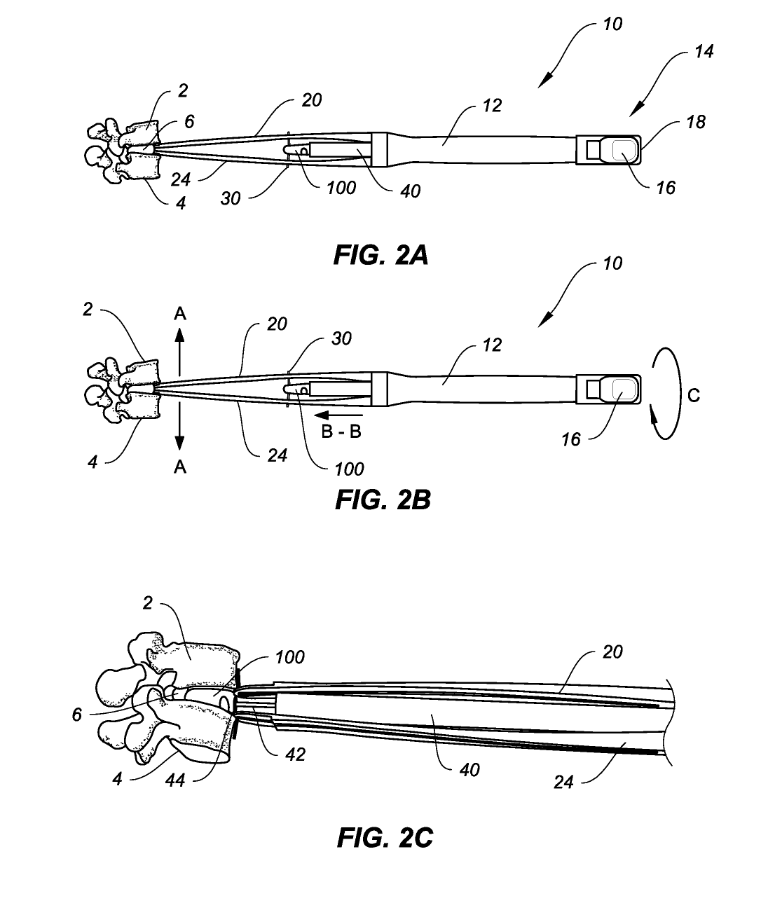

FIGS. 2A to 2C show the stages of the implant 100 being inserted into a disc space between adjacent vertebral bodies 2, 4. As shown in FIG. 2A, the tip region 22, 26 of the instrument 10 can be placed next to the intervertebral space and more specifically next to the disc material 6 in between the vertebral bodies 2, 4. FIG. 2B illustrates one method of actuating the instrument 10. As shown, in one embodiment the user may rotate the T-grip 16 along arc C, thereby causing the translation of the motion arm 40. Translational movement of the motion arm 40 as it extends along lines B-B consequently moves the implant 100 towards the insertion site until guide posts 30 reach the boundaries of the vertebral bodies. Guide posts 30 may thus serve to provide an indication of the amount of insertion and to prevent overinsertion of the implant 100 into the disc space, such as when guide posts 30 reach the end of guide slots 28. In some embodiments, the guide posts 30 may be marked or colored such that the amount of insertion can be readily seen by a user or surgeon. The elongate paddles 20, 24 may also comprise markings or other distance indicators to indicate an amount of insertion by the implant 100. Furthermore, handle 12 may also comprise an indicator (not shown), such as a marker moving along a transparent window in handle 12, to show the user an amount of insertion of implant 100.

As the motion arm 40 progresses (in response to rotation of the T-grip 16), paddles 20, 24 extend open along lines A-A and thereby distract vertebral bodies 2, 4. The notches 32 on the paddles 20, 24 can serve to provide tactile feedback or provide an interference to prevent overextension of the paddles 20, 24 into the intervertebral space.

The motion of T-grip 16 may also comprise features to ensure a user has selected a desired direction of motion. For example, T-grip 16 may provide a ratcheting feature and selector knob or lever (not shown) that the user or surgeon selects for a desired direction. Once a particular direction has been selected, the reverse motion of T-grip 16 may be prevented to confirm to the user that a particular direction has been selected. The ratcheting feature may also be used to allow the user to repeatedly use an arc of motion rather than a full circular motion to cause movement of the implant 100. In other embodiments, the T-grip 16 may comprise a quick release or other release feature that allows the user to quickly retract the implant 100.

In yet other embodiments, the instrument 10 may rely on linear motion rather than rotational motion to insert implant 100. For example, the instrument 10 may comprise a push rod type handle that the user pushes or pulls to control the insertion or retraction of the implant 100.

FIG. 2C shows insertion of the implant 100 between vertebral bodies 2, 4. As can be seen, the motion arm 40 further includes an attachment arm 42 that mates with the implant 100 at terminal end 44. The attachment arm 42 may mate with implant 100 with a variety of engagement mechanisms.

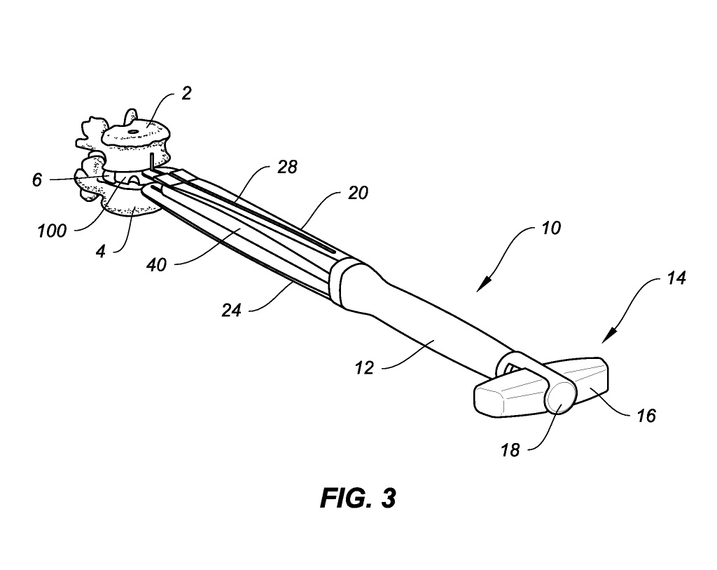

For example, the implant 100, once inserted, may be held in place by natural compression, thus allowing the user to release the attachment arm 42 from the implant 100. In other embodiments, the attachment arm 42 may engage implant 100 with a locking tab or threaded connector (not shown). Once implanted, the user may then manipulate T-grip 16 or other feature of instrument 10 to cause this engagement mechanism to release or unwind. FIG. 3 shows yet another view of the same process whereby the implant 100 has been inserted in place using the instrument 10 of the present invention.

Other embodiments of the invention will be apparent to those skilled in the art from consideration of the specification and practice of the disclosure provided herein. For example, the mechanism for actuating the motion arm 40 may comprise a lever, tensioned cable, a push-pull rod, etc. Further, in some embodiments, the paddles 20, 24 may be detachable and may be provided in a variety of sizes (i.e., lengths) and with different tensile strengths. The paddles 20, 24 may also include a variety of surface features or treatments to facilitate the distraction of the bone tissue of the vertebral bodies 2, 4. The shape of the tip regions 22, 26 may also be differently shaped, such as convex, V-shaped, etc. and may also be enlarged with respect to the rest of the paddles 20, 24. Additionally, the paddles 20, 24 may be curved so as to allow an angular or curved entry to reach the target site. The handle portion 14 may have any variety of grips and surfaces.

What is claimed is:

1. An instrument comprising:
a pair of elongate paddles configured for insertion in between a pair of adjacent vertebral bodies, each paddle being attached at one end to a control handle and terminating at an opposite end in a tapered tip; and
a movable arm positioned in between the elongate paddles, the movable arm being attached at one end to the control handle and being configured at an opposite end to hold a spinal fusion implant for insertion into a disc space between the adjacent vertebral bodies, the movable arm residing between the elongate paddles;
wherein the instrument distracts the vertebral bodies with the elongate paddle as the spinal fusion implant is being delivered to the disc space with the movable arm.

2. The instrument of claim 1, wherein the elongate paddles further include insertion stops for gauging depth of insertion.

3. The instrument of claim 2, wherein the insertion stops prevent overinsertion of the instrument into the disc space.

4. The instrument of claim 2, wherein the insertion stops are raised notches.

5. The instrument of claim 1, wherein the tapered tip of each paddle prevents overdistraction of the disc space.

6. The instrument of claim 1, wherein the elongate paddles include guide slots for receiving guide posts extending from the movable arm.

7. The instrument of claim 6, wherein the guide posts prevent overinsertion of the fusion implant into the disc space.

8. The instrument of claim 1, wherein actuation of the control handle effects translational movement of the movable arm.

9. The instrument of claim 8, wherein the translational movement of the movable arm causes opening or closing of the elongate paddles.

10. The instrument of claim 9, wherein translational movement of the movable arm causes simultaneous opening or closing of the elongate paddles.

11. The instrument of claim 9, wherein translational movement of the movable arm towards the tapered tips of the elongate paddles opens the elongate paddles and distracts the vertebral bodies.

12. The instrument of claim 9, wherein translational movement of the movable arm away from the tapered tips of the elongate paddles closes the elongate paddles.

13. The instrument of claim 1, wherein the control handle comprises a rotatable grip.

14. A method for inserting a spinal fusion implant comprising:
providing a spinal fusion implant;
providing an insertion instrument for delivering the spinal fusion implant, the instrument having a pair of elongate paddles configured for insertion in between a pair of adjacent vertebral bodies, each paddle being attached at one end to a control handle and terminating at an opposite end in a tapered tip, a movable arm positioned in between the elongate paddles, the movable arm being attached at one end to the control handle and being configured at an opposite end to hold a spinal fusion implant for insertion into a disc space between the adjacent vertebral bodies, the movable arm residing between the elongate paddles, wherein the instrument distracts the vertebral bodies with the elongate paddle as the spinal fusion implant is being delivered to the disc space with the movable arm; and
inserting the spinal fusion implant into the disc space with the insertion instrument.

15. The method of claim 14, further including actuating the control handle to effect translational movement of the movable arm.

16. The method of claim 14, wherein translational movement of the movable arm results in subsequent opening or closing of the elongate paddles.

17. The method of claim 16, further including actuating the control handle to translate the movable arm towards the tapered tips of the elongate paddles and causing the elongate paddles to open and distract the vertebral bodies.

18. The method of claim 16, further including actuating the control handle to translate the movable arm away from the tapered tips of the elongate paddles and causing the elongate paddles to close.

19. The method of claim 14, wherein the elongate paddles further include insertion stops for gauging depth of insertion, and further including inserting the tapered tips of the elongate paddles in between the vertebral bodies until the insertion stops reach the vertebral bodies.

20. The method of claim 14, wherein the elongate paddles include guide slots for receiving guide posts extending from the movable arm, and further including translating the movable arm toward the tapered tips of the elongate paddles until the guide posts reach the vertebral bodies.
